(19) United States
(12) Patent Application Publication
(10) Pub. No.: US 2005/0187633 A1
(43) Pub. Date: Aug. 25, 2005

(54) Modular artificial disc replacements (ADRS) that allow translocation and axial rotation

(76) Inventor: Bret A. Ferree, Cincinnati, OH (US)

Correspondence Address: John G. Posa;Gifford, Krass, Groh, Sprinkle,, Anderson & Citokowski, P.C., 280 N. Old Woodward Ave., Suite 400, Birmingham, MI, 48009-5394, US

(21) Appl. No.: 10/987,864

(22) Filed: Nov. 10, 2004

Related U.S. Application Data
(60) Provisional application No. 60/530,579, filed on Dec. 18, 2003.
(60) Provisional application No. 60/518,971, filed on Nov. 10, 2003.

(51) Int. Cl. A61F 2/44

(52) U.S. Cl. 623/17.15

(57) ABSTRACT

Restricted-motion ADRs accommodate translation and/or axial rotation. In preferred embodiments, a portion of the ADR, including the articulating surfaces, is separated, allowing modular component(s) to rotate and/or translate on the ADR endplate (EP). Modularity also permits the use of more than one material. For example, the ADR EP could be made of titanium or chrome cobalt and the articulating component could be made of ceramic or polyethylene. Other materials and other combinations could also be used. The invention could be incorporated into one or both sides of the ADR.

### REFERENCE TO RELATED APPLICATIONS

This application claims priority from U.S. Provisional Patent Application Ser. Nos. 60/518,971, filed Nov. 10, 2003, and 60/530,579, filed Dec. 18, 2003, the entire content of both of which are incorporated herein by reference.

### FIELD OF THE INVENTION

This invention relates generally to artificial disc replacements (ADRs) and, in particular, to modular and restricted-motion ADRs to accomodate translation and/or axial rotation.

### BACKGROUND OF THE INVENTION

Many spinal conditions, including degenerative disc disease, can be treated by spinal fusion or through artificial disc replacement (ADR). ADR has several advantages over spinal fusion. The most important advantage of ADR is the preservation of spinal motion. Spinal fusion eliminates motion across the fused segments of the spine. Consequently, the discs adjacent to the fused level are subjected to increased stress. The increased stress increases the changes of future surgery to treat the degeneration of the discs adjacent to the fusion. However, motion through an ADR also allows motion through the facet joints. Motion across arthritic facet joints could lead to pain following ADR. Some surgeons believe patients with degenerative disease and arthritis of the facet joints are not candidates for ADR.

Current ADR designs do not attempt to limit the pressure across the facet joints or facet joint motion. Indeed, prior art ADRs generally do not restrict motion. For example, some ADR designs place bags of hydrogel into the disc space which do not limit motion in any direction. In fact, ADRs of this kind may not, by themselves, provide sufficient distraction across the disc space. ADR designs with metal plates and polyethylene spacers may restrict translation but they do not limit the other motions mentioned above. The articular surface of the poly spacer is generally convex in all directions. Some ADR designs limit motion translation by attaching the ADR halves at a hinge.

One of the most important features of an artificial disc replacement (ADR) is its ability to replicate the kinematics of a natural disc. ADRs that replicate the kinematics of a normal disc are less likely to transfer additional forces above and below the replaced disc. In addition, ADRs with natural kinematics are less likely to stress the facet joints and the annulus fibrosus (AF) at the level of the disc replacement. Replicating the movements of the natural disc also decreases the risk of separation of the ADR from the vertebrae above and below the ADR.

### SUMMARY OF THE INVENTION

This invention enables restricted-motion ADRs to accomodate translation and/or axial rotation. In preferred embodiments, a portion of the ADR, including the articulating surfaces, is separated, allowing modular component(s) to rotate and/or translate on the ADR endplate (EP). Modularity also permits the use of more than one material. For example, the ADR EP could be made of titanium or chrome cobalt and the articulating component could be made of ceramic or polyethylene. Other materials and other combinations could also be used. The invention could be incorporated into one or both sides of the ADR.

### BRIEF DESCRIPTION OF THE DRAWINGS

FIG. 1A is a sagittal cross section of an ADR constructed in accordance with the present invention;

FIG. 1B is an exploded view of the ADR shown in FIG. 1A;

FIG. 1C is a view of the articulating side of one of the ADR EPs;

FIG. 1D is a view of the articulating side of an alternative embodiment of the ADR EP shown in FIG. 1C;

FIG. 1E is a view of the ADR side of the modular articulating component;

FIG. 1F is a view of the ADR side of an alternative embodiment of the modular articulating component shown in FIG. 1E;

FIG. 2A is a sagittal cross section of an alternative embodiment of the ADR;

FIG. 2B is a view of the articulating surface of the modular component and the articulating side of the ADR EP;

FIG. 3A is a sagittal cross-section of another embodiment of the present invention;

FIG. 3B is an axial cross section through the ADR drawn in FIG. 3A;

FIG. 3C shows a circular projection from the ADR EP cooperating with the circular opening;

FIG. 4 is a sagittal cross-section of another alternative embodiment of the present invention;

FIG. 5A is a lateral view of the spine and an ADR according to the present invention;

FIG. 5B is a lateral view of the embodiment of the ADR drawn in FIG. 5A;

FIG. 6A is a sagittal cross-section of a further alternative embodiment of the present invention; and

FIG. 6B is a view of the superior surface of the intermediate component shown in FIG. 6A.

### DETAILED DESCRIPTION OF THE INVENTION

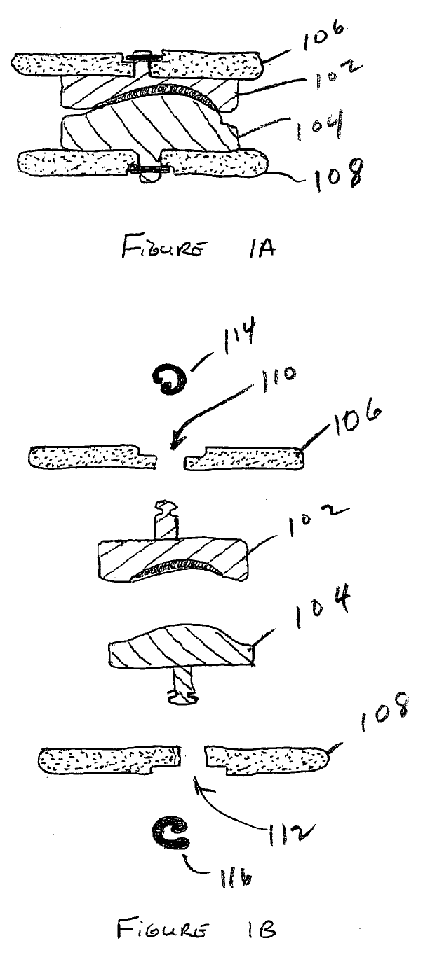

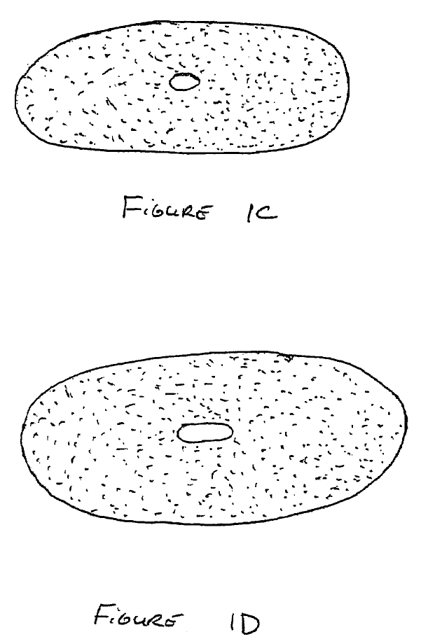

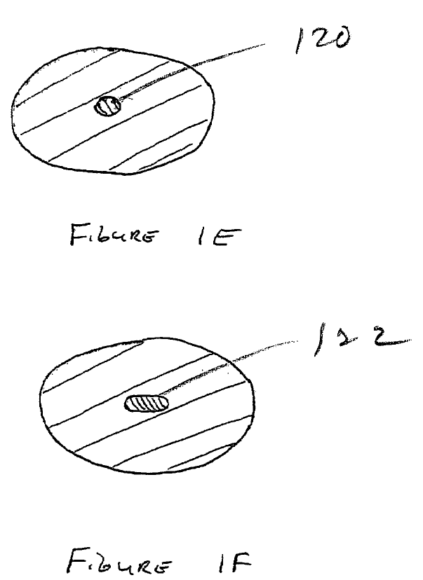

FIG. 1A is a sagittal cross section of an ADR constructed in accordance with the invention, wherein modular articulating components 102, 104 articulate with one another and with their respective ADR EPs 106, 108. FIG. 1B is an exploded view of the ADR drawn in FIG. 1A. The modular articulating components fit through holes 110, 112 in the ADR EPs. C-rings 114, 116 or other devices can be used to hold the modular articulating components in the ADR EPs.

FIG. 1C is a view of the articulating side of one of the ADR EPs. The central hole permits axial rotation of the modular articulating component. FIG. 1D is a view of the articulating side of an alternative embodiment of the ADR EP drawn in FIG. 1C. An oblong central hole permits axial rotation and translation of the modular articulating component. FIG. 1E is a view of the ADR side of the modular articulating component. The circular shaft 120 of the projection from the modular component permits unlimited axial rotation.

FIG. 1F is a view of the ADR side of an alternative embodiment of the modular articulating component drawn in FIG. 1E. A shaft 122 having an oblong cross section from the modular component cooperates with the hole in the ADR EP drawn in FIG. 1D to allow limited translation and limited rotation. For example, the shaft and hole could be configured to allow 2-5 degrees of axial rotation and 1-3 mm of translation.

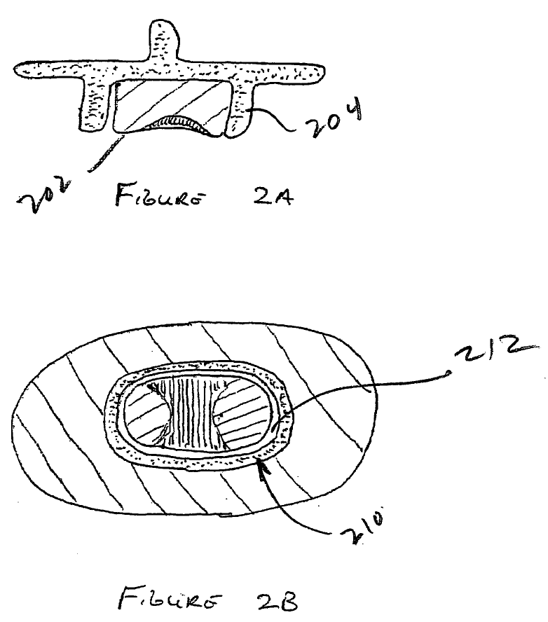

FIG. 2A is a sagittal cross section of an alternative embodiment of the ADR. The modular articulating component 202 fits into an opening in the ADR EP 204. The difference in the size and shape of the modular articulating component and the opening in the ADR EP determine the type and amount of motion permitted between the two components. For example, the components could be sized and shaped to permit 1-3 mm of translation and 2-5 degrees of axial rotation. FIG. 2B is a view of the articulating surface of the modular component and the articulating side of the ADR EP. The area 210 of the drawing represents the wall of the ADR EP that contains the modular articulating component. The space between the modular articulating component and the retaining wall of the ADR EP is shown at 212.

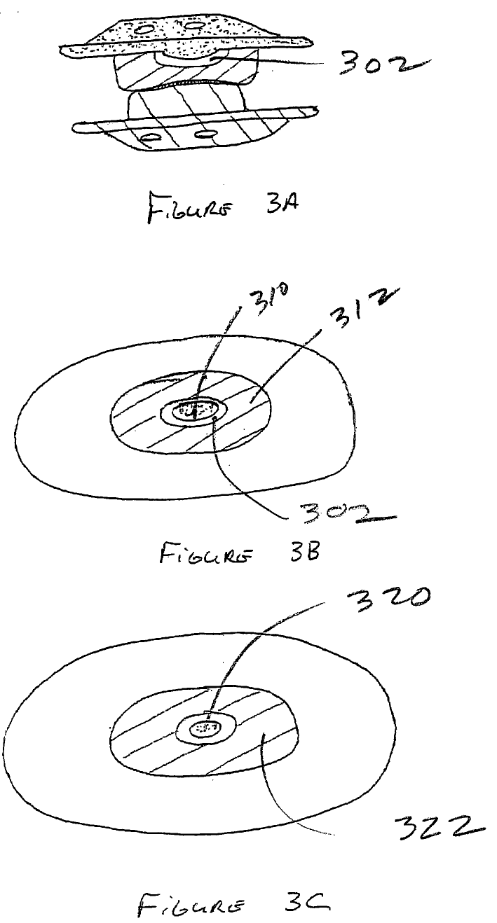

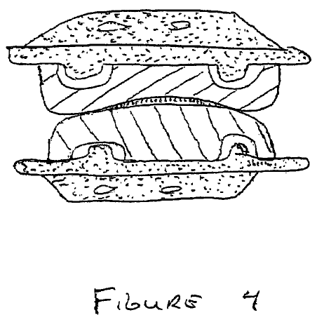

FIG. 3A is a sagittal cross section of another embodiment wherein a recess in the modular articulating component cooperates with a raised area on the ADR EP to allow axial rotation and translation. The area 302 between the modular articulating component and the raised area on the superior ADR EP represents the space between the two components. FIG. 3B is an axial cross section through the ADR drawn in FIG. 3A at the level of the raised projection form the superior ADR EP. The oval shaped projection 310 from the ADR EP cooperates with the oval opening 312 in the modular articulating component to allow limited rotation and limited translation. Alternatively, a circular shaped projection from the ADR EP could cooperate with an oval shaped recess in the modular component to allow unlimited axial rotation and/or increased translation. As shown in FIG. 3C, a circular projection 320 from the ADR EP cooperates with the circular opening 322 in the modular component to allow unlimited axial rotation and limited translocation.

FIG. 4 is a sagittal cross section of an alternative embodiment of the invention wherein two or more projections from the ADR EPs cooperate with two or more recesses in the modular articulating components. The use of two or more projections limits axial rotation.

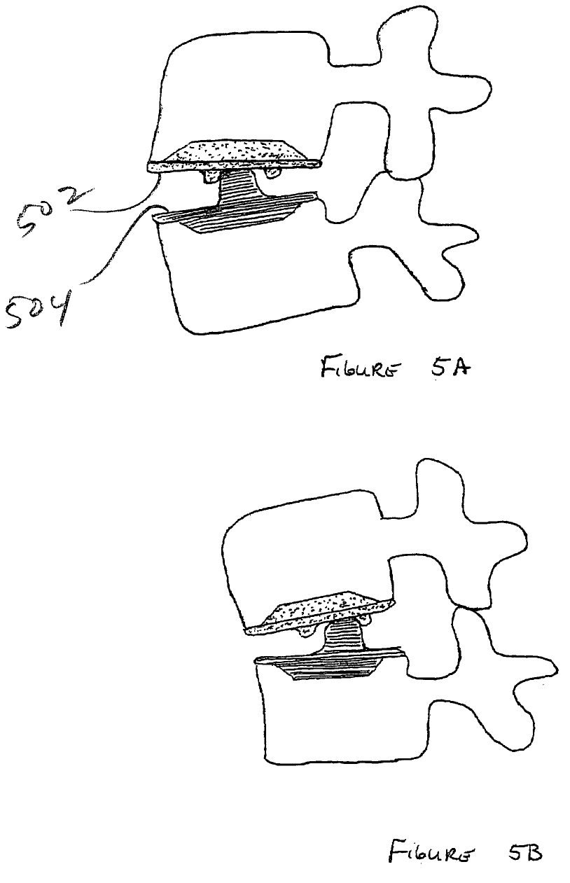

FIG. 5A is a lateral view of the spine and an ADR according to the invention with components that cooperate to prevent posterior translation yet allow anterior translation. Impingement between the ADR components prevent the superior ADR Endplate (EP) 502 from translating posteriorly on the inferior ADR EP 504. The superior ADR EP may impinge on the inferior ADR EP to prevent excessive anterior translocation. FIG. 5B is a lateral view of the embodiment of the ADR drawn in FIG. 5A and a spine in flexion, showing how the superior ADR EP translates and flexes on the inferior ADR EP.

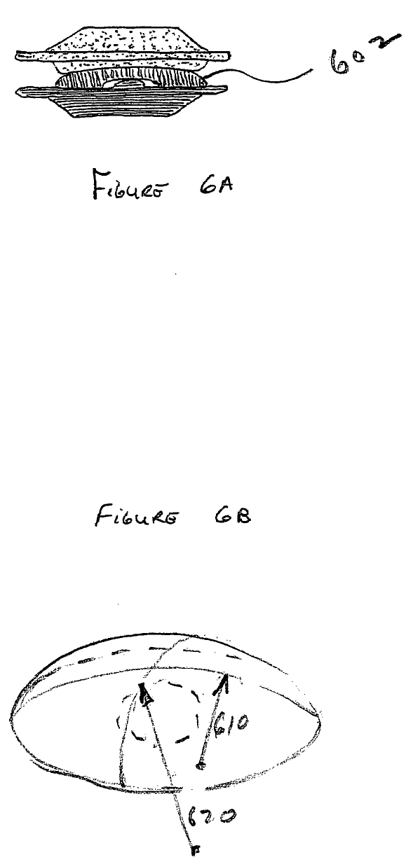

FIG. 6A is a sagittal cross section of a further alternative embodiment, wherein the superior surface of the intermediate component 602 is made by curves of at least two different radii. For example, the anterior-posterior curve may be formed by a radius 610 of 20 mm and the left-right curve could be formed by a radius 620 of 40 mm. Other radii could be used, including an anterior-posterior curve larger that the left-right curve, depending upon spinal level and other factors. The spherical surface of the superior portion of the intermediate component permits flexion, extension, and lateral bending. The different radii of the superior surface of the intermediate component inhibit axial rotation. The flat surface of the intermediate component articulates with the flat surface of the inferior ADR EP to permit axial rotation and translation of the ADR.

FIG. 6B is a view of the superior surface of the intermediate component drawn in FIG. 6A. The different radii of the curves of the spherical superior surface of the component give the component an oblong shape. For example, the component could be 20 mm as measured from anterior to posterior and 40 mm as measured from left to right.

1. An artificial disc replacement (ADR), comprising:
an endplate component adapted for attachment to a vertebral body or endplate; and
a spacer component having an articular surface and an interface to the endplate component that facilitates at least a limited amount of translation or axial rotation of the spacer component independent of the articular surface.

2. The ADR of claim 1, wherein the interface includes a projection located in an aperture or depression slightly larger than the projection to facilitate at least a limited amount of translation, axial rotation, or both.

3. The ADR of claim 2, wherein the projection extends from the spacer component and the aperture or depression is in the endplate component.

4. The ADR of claim 2, wherein the projection extends from the endplate component and the aperture or depression is in the spacer component.

5. The ADR of claim 2, wherein the post and the aperture are round.

6. The ADR of claim 2, wherein the post and the aperture are oblong.

7. The ADR of claim 2, wherein the post is round and the aperture is oblong.

8. The ADR of claim 2, including a plurality of projections and corresponding apertures or depressions.

9. The ADR of claim 1, wherein the articular surface is convex.

10. The ADR of claim 9, wherein the articular surface is composed of multiple radii of curvature.

11. The ADR of claim 1, further including:
a second endplate component adapted for attachment to an opposing vertebral body or endplate; and
wherein the articular surface cooperates with a corresponding articular surface on an second endplate.

12. The ADR of claim 11, wherein the articular surface and the corresponding articular surface form a saddle-shaped joint.

13. The ADR of claim 1, further including:
a second endplate component adapted for attachment to an opposing vertebral body or endplate; and
a second spacer component coupled to the second endplate component.

14. The ADR of claim 13, wherein the second spacer component includes an articular surface and an interface to the second endplate component that facilitates at least a limited amount of translation or axial rotation of the second spacer component independent of the articular surface.

15. The ADR of claim 14, wherein the interface includes a projection located in an aperture or depression slightly larger than the projection to facilitate at least a limited amount of translation, axial rotation, or both.

16. The ADR of claim 15, wherein the projection extends from the second spacer component and the aperture or depression is in the second endplate component.

17. The ADR of claim 15, wherein the projection extends from the second endplate component and the aperture or depression is in the second spacer component.

18. The ADR of claim 1, further including:
a second endplate component adapted for attachment to an opposing vertebral body or endplate;
a second spacer component coupled to the second endplate component; and
wherein the second spacer component includes an articular surface that cooperates with the articular surface on an endplate component.

19. The ADR of claim 18, wherein the articular surfaces of the spacer components form a saddle-shaped joint.
